(19) United States
(12) Patent Application Publication
(10) Pub. No.: US 2022/0273578 A1
(43) Pub. Date: Sep. 1, 2022

(54) FloraTummys Probiotic Quick Dissolve Tablet

(71) Applicant: Frank Melfa, North Bergen, NJ (US)

(72) Inventor: Frank Melfa, North Bergen, NJ (US)

(21) Appl. No.: 17/155,135

(22) Filed: Mar. 1, 2021

(51) Int. Cl. A61K 9/20

(52) U.S. Cl. CPC A61K 9/2072; A61K 9/2018; A61K 9/2013; A61K 2035/115

(57) ABSTRACT

A tablet composition containing any probiotic and or prebiotic in combination with isomalt, calcium palmitate or any dissolving agent that allows the tablet to dissolve within minutes when mixed with liquids for the use in the prevention of cross contamination of probiotics in hospitals or healthcare facilities where probiotics are administered.

### BACKGROUND OF INVENTION

Hospital studies have reported cross contamination †(Cross Colonization) of probiotics in their hospital units, particularly when using powder probiotics which are commonly used in preterm babies in the neonatal intensive care unit. Probiotics have proven to reduce a fatal infection called necrotizing enterocolitis or (NEC) in premature babies. †Gengaimuthu, K: The Cross Contamination (Cross Colonization) Phenomenon of Probiotic Use in NICUs: Neonatal and Pediatrics, International Modern Hospital, Dubai, ARE. May 2018. DOI: 10.7759/cureus.2691

Although probiotics have proven to reduce NEC infection and death from NEC, some hospitals choose not to use probiotics because they have shown to contaminate the units. As a result, premature babies in these hospitals are being denied a proven therapy to reduce NEC infection and death.

The problem of †Cross Contamination (Cross Colonization) of probiotics starts when a healthcare provider opens a powder packet or bulk powder container in a hospital setting. The particles escape in the air and spread and attach to surfaces, food, and passed onto patients. Probiotics are living organisms that can be spread through the hospital and consumed and inhaled by patients, contaminating the hospital units. This is a problem and I believe I have developed a novel solution with FloraTummys Quick Dissolve Tablets.

### BRIEF SUMMARY OF INVENTION/SOLUTION

FloraTummys Quick Dissolve Tablets are the only probiotic tablets on the market that completely dissolves in liquids within minutes and that can help reduce the incidence of cross contamination of probiotics in hospital or healthcare facilities that use probiotics.

FloraTummys Quick Dissolve Probiotic Tablets can help solve the problem of cross contamination or †Cross Colonization by preventing powder particles from dispersing in the air particularly in the NICU (Neonatal Intensive Care Unit) where powdered probiotics are used for preterm infants. Powdered probiotics are often mixed in any part of the hospital or nursing home when a patient cannot ingest food.

A FloraTummys Quick Dissolve Tablet is placed into a syringe or closed container and then mixed or shaken until it completely dissolves. This lowers the chance of powder particles escaping in the air. It takes the right amount of probiotics, prebiotics, combined and formed with other ingredients specifically dissolving agents and a tablet finish to make this tablet dissolve.

### DETAILED DESCRIPTION OF THE INVENTION

There are many probiotics on the market and several in tablet form. Most tablet probiotics are designed to be chewed and usually flavored to make it palatable. There are also probiotics that dissolve in the mouth but not designed to dissolve in liquids.

The tablet, in addition to its ability to completely dissolve in liquids must be a finished tablet with a smooth coating which also helps prevent particles from escaping in the air that cause cross contamination.

Powder probiotics when opened from a packet or container allow powder particles to immediately escape in the air. FloraTummys Quick Dissolve Tablet is placed in a syringe or closed container and then mixed with liquids.

The combination of probiotics, prebiotics, tablet lubricants, and sugar alcohol in FloraTummys allows for the tablet to completely dissolve in minutes. Isomalt is the sugar alcohol or dissolving agent in FloraTummys that allows the tablet to dissolve within minutes when mixed with liquids. The lubricant in FloraTummys that coats the tablet is calcium palmitate.

When mixed with liquids such as water, breastmilk, or formula the FloraTummys Quick Dissolve Tablet dissolves within minutes with little to no likelihood of allowing powder particles from escaping into the air like powder probiotics.

The ingredients in FloraTummys Quick Dissolve Tablets include: Probiotics (Bifidobacterium infantis and Lactobacillus reuteri), prebiotic (fructooligosaccharide), Isomalt, Calcium Palmitate.

1. FloraTummys Quick Dissolve Tablets are the first and only probiotics/prebiotic tablets designed to dissolve when mixed with liquids and marketed to help hospitals and other healthcare facilities to reduce the potential incidence of cross contamination of probiotics.
I am not looking to patent the FloraTummys probiotic prebiotic combination of Bifidobacterium infantis, Lactobacillus reuteri or any probiotic/prebiotic combination, as there are many probiotics on the market with Bifidobacterium infantis, Lactobacillus reuteri with or without a prebiotic.
I'm looking to patent any probiotic or prebiotic tablet that dissolves in liquids and marketed to dissolve in liquids. I am also looking to patent any probiotic or prebiotic that dissolves in liquids for the purpose of helping reduce the potential of cross contamination of probiotics in hospitals or any healthcare facility that use probiotics.
If possible, I would want to patent the combination of any probiotic or prebiotic in combination with isolmalt and calcium palmitate. As this combination is specifically designed to dissolve in liquids and prevents probiotic powder particles from being released in the air.
